(19) United States
(12) Patent Application Publication
(10) Pub. No.: US 2024/0408255 A1
(43) Pub. Date: Dec. 12, 2024

(54) ODOR CONTROL COMPOSITIONS INCLUDING ESSENTIAL OIL(S) AND HIGH SURFACE ESSENTIAL MINERALS (HSAEM)

(71) Applicant: MICROBAN PRODUCTS COMPANY, Huntersville, NC (US)

(72) Inventors: Daniel BATES, Charlotte, NC (US); Ryan Scott, Charlotte, NC (US); Ishmell Williams, Charlotte, NC (US); Lillian Stephens, Charlotte, NC (US)

(21) Appl. No.: 18/737,172

(22) Filed: Jun. 7, 2024

Related U.S. Application Data
(60) Provisional application No. 63/471,568, filed on Jun. 7, 2023.

(51) Int. Cl. A61L 2/18; A61L 101/32; C11D 3/00

(52) U.S. Cl. CPC A61L 2/18; C11D 3/0068; A61L 2101/32; A61L 2202/26

(57) ABSTRACT

An odor control composition including an essential oil that binds and/or neutralizes organic malodor molecules; and a high surface area essential mineral (HSAEM) that binds and/or neutralize organic malodor molecules. Textiles having the odor control compositions applied thereon and/or treated with the odor control compositions in which the treated textile binds and/or neutralizes the organic malodor molecules for a prolonged period of time (e.g., up to twenty-five (25) wash cycles, for several months, for several years, or any combination thereof).

### TECHNICAL FIELD

The present invention relates generally to the field of odor control compositions and textiles treated with the same, and more particularly, odor control compositions including an essential oil that binds and/or neutralizes organic malodor molecules; and a high surface area essential mineral (HSAEM) that binds and/or neutralize organic malodor molecules. In certain aspects also disclosed are textiles having the odor control compositions applied thereon and/or treated with the odor control compositions in which the treated textile binds and/or neutralizes the organic malodor molecules for a prolonged period of time (e.g., up to twenty-five (25) wash cycles, for several months, for several years, or any combination thereof).

### BACKGROUND

The current field of textile odor reduction on textile materials (e.g., dry textile materials and articles) has a limited number of options and formulations, which mostly utilize heavy metals or nanomaterials to reduce specific odors. These heavy metal based formulations and nanomaterials have many drawbacks, which include being environmentally harsh. In particular, these materials are typically used in conjunction with a polymer/polymer blend to adhere to textiles, but overtime these metals wash out of the textiles and leach into the soil and ground water, which, in high concentrations, are harmful to plants and animals.

### SUMMARY

Accordingly, there is a need to provide environmentally friendly odor control compositions/formulations that avoid the use of heavy metals and nanomaterials. In certain aspects, disclosed herein are environmentally friendly odor control compositions/formulations that are just as effective, if not more effective, than the currently known heavy metal and nanomaterial formulations. The compositions, articles, and methods disclosed herein overcome the above-mentioned problems observed with heavy metal and nanomaterial compositions by providing environmentally friendly compositions that utilize a combination of essential oils and high surface area essential minerals that synergistically interact with one another to reduce (by binding and neutralizing) certain organic malodors (i.e., odors often associated with human body odor including odorous human secretions and/or by-product produced by bacteria and/or yeast found on the human body) on textile materials for a prolonged period of time. The organic malodors include, for example, isovaleric acid, ammonia, acetic acid, and nonenal.

Moreover, and unlike the above-mentioned heavy metals and nanomaterial compositions, the compositions disclosed herein remain within a textile longer than formulations that include heavy metals and nanomaterials because the disclosed compositions are wash resistant. This wash resistance advantageously allows for the disclosed compositions to remain in a textile for prolonged periods of time (e.g., 25 wash cycles, several months, or up to a year) and impart odor control in and on the textile reducing odors associated with isovaleric acid, ammonia, acetic acid, and nonenal. Without wishing to be bound by theory, it is thought that the insolubility of the essential oils and high surface area of the HSAEM(s) prevent the disclosed compositions from washing out of the textile material/textile fabric thereby allowing for repeated uses and prolonged odor control for textile materials having the disclosed compositions applied thereon.

In certain aspects, disclosed is an odor control composition comprising: (a) an essential oil (at an effective amount) that binds and/or neutralizes organic malodor molecules; and (b) a high surface area essential mineral (HSAEM) (at an effective amount) that binds and/or neutralize organic malodor molecules. The essential oils disclosed herein advantageously function to bind and/or neutralize the organic molecules while concurrently functioning to adhere the HSAEM to the textile material. In certain aspects, the composition may further include water therein (e.g., when the composition is a water in oil emulsion), and when included, water is present at a concentration ranging from 10 wt % to 30 wt % of the composition in which any endpoint falling therein may serve as an endpoint for additional ranges, additional ranges may include, for example, from 15 wt % to 27.5 wt %, from 15 wt % to 25 wt %, from 17.5 wt % to 25 wt %,

In certain aspects, the essential oil is present in the odor control composition (before application to the textile) at a concentration ranging from 25 wt % of the composition to 65 wt % of the composition in which any endpoint falling therein may serve as an endpoint for additional ranges, additional ranges may include, for example, from 25 wt % to 60 wt %, from 30 wt % to 55 wt %, from 35 wt % to 55 wt %, from 40 wt % to 50 wt %.

In certain aspects, the HSAEM is present in the odor control composition (before application to the textile) at a concentration ranging from 10 wt % of the composition to 50 wt % of the composition in which any endpoint falling therein may serve as an endpoint for additional ranges, additional ranges may include, for example, from 15 wt % to 50 wt %, from 20 wt % to 45 wt %, from 25 wt % to 40 wt %, from 30 wt % to 40 wt %.

In certain aspects, the essential oil and HSAEM are present at a ratio of wherein the essential oil and HSAEM are present at a ratio of 3:1 to 1:3, 2:1 to 1:2 more preferably 1.5:1 to 1:1.5, most preferably 1:1.

In certain aspects, the essential oil comprises aloe oil, castor oil, hemp seed oil, flax seed oil, or any combination thereof. In certain aspects, the essential oil comprises at least two of aloe oil, castor oil, hemp seed oil, flax seed oil, and canola oil. In certain aspects, essential oil comprises at least three of aloe oil, castor oil, hemp seed oil, flax seed oil, and canola oil. In certain aspects, the essential oil comprises aloe oil, castor oil, hemp seed oil, flax seed oil, and canola oil. When there are two essential oils present in the compositions, the oils may be present in a 5:1 to 1:5 ratio, a 3:1 to 1:3 ratio, or a 1:1 ratio.

In certain aspects, the HSAEM is a clay material. In certain aspects, the HSAEM is a zeolite.

In certain aspects, the HSAEM and more particularly zeolite is chabazite and/or pentasil.

In certain aspects, the composition is a liquid. In certain aspects, the composition is an emulsion.

In certain aspects, the composition is a water in oil emulsion that is configured to be applied to a textile and/or to treat a textile and subsequently dried/cured thereon to impart prolonged odor control (e.g., a predetermined number of wash cycles including 10 wash cycles and/or 25 wash cycles and/or a predetermined period of time for several months to several years post-treatment and drying/curing on the textile).

In certain aspects, wherein the organic malodor molecules comprise ammonia, acetic acid, isovaleric acid, nonenal, or any combination thereof.

In certain aspects, the organic malodor molecules comprise at least two of ammonia, acetic acid, nonenal, and isovaleric acid.

In certain aspects, the organic malodor molecules comprises ammonia, acetic acid, nonenal, and isovaleric acid.

Also disclosed herein are textile coating compositions comprising: (a) an essential oil (at an effective amount) that binds and/or neutralizes organic odor molecules (effective amount(s) to bind and/or neutralize organic odor molecules); and (b) a high surface area essential mineral (HSAEM) (at an effective amount) that binds and/or neutralizes organic odor molecules (effective amount(s) to bind and/or neutralize organic odor molecules). The essential oils disclosed herein advantageously function to bind and/or neutralize the organic molecules while concurrently functioning to adhere the HSAEM to the textile material. In certain aspects, the composition may further include water therein (e.g., when the composition is a water in oil emulsion), and when included, water is present at a concentration ranging from 10 wt % to 30 wt % of the composition in which any endpoint falling therein may serve as an endpoint for additional ranges, additional ranges may include, for example, from 15 wt % to 27.5 wt %, from 15 wt % to 25 wt %, from 17.5 wt % to 25 wt %.

In certain aspects, the essential oil is present in the odor control composition (before application to the textile) at a concentration ranging from 25 wt % of the composition to 65 wt % of the composition in which any endpoint falling therein may serve as an endpoint for additional ranges, additional ranges may include, for example, from 25 wt % to 60 wt %, from 30 wt % to 55 wt %, from 35 wt % to 55 wt %, from 40 wt % to 50 wt %.

In certain aspects, the HSAEM is present in the odor control composition (before application to the textile) at a concentration ranging from 10 wt % of the composition to 50 wt % of the composition in which any endpoint falling therein may serve as an endpoint for additional ranges, additional ranges may include, for example, from 15 wt % to 50 wt %, from 20 wt % to 45 wt %, from 25 wt % to 40 wt %, from 30 wt % to 40 wt %.

In certain aspects, the essential oil and HSAEM are present at a ratio of 3:1 to 1:3, 2:1 to 1:2 more preferably 1.5:1 to 1:1.5, most preferably 1:1.

In certain aspects, the essential oil comprise aloe oil, castor oil, hemp seed oil, flax seed oil, canola oil, or any combination thereof. In certain aspects, the essential oil comprises at least two of aloe oil, castor oil, hemp seed oil, flax seed oil, and canola oil. In certain aspects, essential oil comprises at least three of aloe oil, castor oil, hemp seed oil, flax seed oil, and canola oil. In certain aspects, the essential oil comprises aloe oil, castor oil, hemp seed oil, flax seed oil, and canola oil. When there are two essential oils present in the compositions, the oils may be present in a 5:1 to 1:5 ratio, a 3:1 to 1:3 ratio, or a 1:1 ratio.

In certain aspects, the HSAEM is a clay material. In certain aspects, the HSAEM is a zeolite.

In certain aspects, the HSAEM is and more particularly zeolite chabazite and/or pentasil.

In certain aspects, the composition is a liquid. In certain aspects, the composition is an emulsion.

In certain aspects, the composition is a water in oil emulsion, containing between 10-30% water in which any endpoint falling therein may serve as an endpoint for additional ranges, that is configured to be applied to a textile and/or to treat a textile and subsequently dried/cured thereon to impart prolonged odor control (e.g., a predetermined number of wash cycles including 10 wash cycles and/or 25 wash cycles and/or a predetermined period of time for several months to several years post-treatment and drying/curing on the textile).

In certain aspects, wherein the organic malodor molecules comprise ammonia, acetic acid, isovaleric acid, nonenal, or any combination thereof.

In certain aspects, the organic malodor molecules comprise at least two of ammonia, acetic acid, nonenal, and isovaleric acid.

In certain aspects, the organic malodor molecules comprises ammonia, acetic acid, nonenal, and isovaleric acid.

Also disclosed herein are textiles having at least one of the above-mentioned odor control compositions applied thereon (and/or treated therewith). In certain aspects, the textiles are subsequently dried and/or cured such that the above-mentioned odor control compositions are permanently adhered thereon and impart prolonged odor control. In certain aspects also disclosed are textiles having the odor control compositions applied thereon and/or treated with the odor control compositions in which the treated textile binds and/or neutralizes the organic malodor molecules for a prolonged period of time (e.g., up to twenty-five (25) wash cycles, for several months, for several years, or any combination thereof). In certain additional aspects, the odor control composition is uniformly and/or homogeneously applied on the textile.

In certain aspects, the essential oil applied on the textile ranges from 1.5% percent weight on fabric (owf) to 6.5% owf, more preferably 1.75% owf to 5% owf, and most preferably 2% owf to 3% owf, and the HSAEM applied on the textile ranges from 0.5% percent weight on fabric (owf) to 6% owf, more preferably 0.5% owf to 5% owf, and most preferably 1% owf to 3% owf.

Also disclosed herein are dry textile material(s) having one of the above-mentioned compositions applied thereon, wherein the dry textile material and/or the composition applied thereon binds and neutralizes organic malodor molecules on the dry textile material for a prolonged period of time. In this aspect, the essential oil applied on the dry textile ranges from 1.5% percent weight on fabric (owf) to 6.5% owf, more preferably 1.75% owf to 5% owf, and most preferably 2% owf to 3% owf, and the HSAEM applied on the dry textile ranges from 1.5% percent weight on fabric (owf) to 6.5% owf, more preferably 1.75% owf to 5% owf, and most preferably 2% owf to 3% owf. In this aspect, the essential oil and HSAEM are present on the dry textile material at a ratio of 3:1 to 1:3, 2:1 to 1:2 more preferably 1.5:1 to 1:1.5, most preferably 1:1. In this aspect, the dry textile material and/or the composition applied thereon binds and neutralizes ammonia, acetic acid, and isovaleric acid on the textile material when compared to an untreated textile material. In this aspect, the dry textile material binds and neutralizes 90% to 100% and more preferably 95% to 100% of ammonia when compared to an untreated textile material, and/or the dry textile material binds and neutralizes 20% to 70% and more preferably 30% to 70% of acetic acid when compared to an untreated textile material, and/or the dry textile material binds and neutralizes 20% to 70% and more preferably 30% to 70% of isovaleric acid when compared to an untreated textile material.

Also disclosed herein are methods of applying the odor control composition to a textile material comprising: applying the odor control composition to the textile material, wherein the odor control composition comprises (a) an essential oil at an effective amount that binds and neutralizes organic odor molecules; and (b) a high surface area essential mineral (HSAEM) at an effective amount that binds and neutralizes odor molecules. In certain aspects, the odor control composition is applied to the textile material via exhausting/an exhaustion method and subsequently dried such that the treated textile binds and/or neutralizes the organic malodor molecules for a prolonged period of time (e.g., up to twenty-five (25) wash cycles, for several months, for several years, or any combination thereof). The exhaustion process consists of adding the essential oils and high surface area essential minerals solution and fabric to an exhaustion machine and it will gradually increase the temperature and gradually decrease the temperature over a desired timeframe. In other aspects, the odor control composition is applied to the textile material via a padding method and subsequently dried such that the treated textile binds and/or neutralizes the organic malodor molecules for a prolonged period of time (e.g., up to twenty-five (25) wash cycles, for several months, for several years, or any combination thereof). The padding process requires applying the essential oils and high surface area essential minerals solution to a fabric and moving it through rollers then drying the fabric in the oven to cure. When the textile is either padded or exhausted with high heat and pressure the essential oils and high surface area essential minerals combination will be incorporated into the fabric (e.g., permanently incorporated) and will remain there for a prolonged period of time (e.g., a predetermined number of wash cycles).

Embodiments of the invention can include one or more or any combination of the above features and configurations.

Additional features, aspects and advantages of the invention will be set forth in the detailed description which follows, and in part will be readily apparent to those skilled in the art from that description or recognized by practicing the invention as described herein. It is to be understood that both the foregoing general description and the following detailed description present various embodiments of the invention, and are intended to provide an overview or framework for understanding the nature and character of the invention as it is claimed. The accompanying drawings are included to provide a further understanding of the invention, and are incorporated in and constitute a part of this specification.

### DETAILED DESCRIPTION

The present invention will now be described more fully hereinafter with reference to the working examples in which exemplary embodiments of the invention are shown. However, the invention may be embodied in many different forms and should not be construed as limited to the representative embodiments set forth herein. The exemplary embodiments are provided so that this disclosure will be both thorough and complete, and will fully convey the scope of the invention and enable one of ordinary skill in the art to make, use and practice the invention.

Further, the term “or” as used in this disclosure and the appended claims is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form. Throughout the specification and claims, the following terms take at least the meanings explicitly associated herein, unless the context dictates otherwise. The meanings identified below do not necessarily limit the terms, but merely provide illustrative examples for the terms. The meaning of “a,” “an,” and “the” may include plural references, and the meaning of “in” may include “in,” “at,” and/or “on,” unless the context clearly indicates otherwise. The phrase “in one embodiment,” as used herein does not necessarily refer to the same embodiment, although it may.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within the ranges as if each numerical value and sub-range is explicitly recited. As an illustration, a numerical range of “about 1 to 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc. as well as 1, 2, 3, 4, and 5, individually. The same principle applies to ranges reciting only one numerical value as a minimum or a maximum. Furthermore, such an interpretation should apply regardless of the breadth of the range or the characteristics being described.

“High surface area essential mineral (HSAEM)” includes a mineral having a specific surface area (SSA) of greater than 250 m²/g (ranges from 250 m²/g to 500 m²/g and more preferably 300 m²/g to 500 m²/g). Examples of HSAEM(s) include, but are not limited to, clay and clay materials, more particularly zeolites, and most particularly chabazite and/or pentasil.

“Owf” or percent on weight of fabric is generally used in the field and batch processes, the amount of chemical finish to be applied is usually expressed as a weight percentage based on the original fabric weight. This relationship is abbreviated as % owf (percent on weight of fabric), which is % OWF=[(Weight of Chemical/Weight of Fabric)*100]/Wet pickup rate %. For example, if a chemical is to be applied at 3% owf to 400 kg of fabric, having a wet pickup rate=80%, then 15 kg of the chemical will be used (3% of 400 kg at 80% pickup).

### Odor Control Compositions And Textile Coatings (Concentrate)

Disclosed herein are odor control compositions and textile coatings provided in the form of concentrates (pre-application to the textile material) that are applied (e.g., subsequently diluted and permanently applied via padding or exhaustion techniques) to and/or used to treat textile materials to reduce and/or neutralize odor and/or organic malodor molecules (secreted and/or excreted from humans) on the textile material for a prolonged period of time (e.g., 25 wash cycles, several months, or up to a year). The disclosed compositions and textile coatings disclosed herein are environmentally friendly odor control compositions/formulations that are just as effective for odor control and reduction, if not more effective, than the currently known heavy metal and nanomaterial formulations on textile materials treated with the disclosed compositions.

In particular, the odor control compositions and textile coatings disclosed herein (provided in the form of a concentrate) include a combination of essential oils and high surface area essential minerals (HSAEMs). The odor control compositions and textile coatings disclosed herein (and provided in the form of a concentrate) may further include water. The odor control compositions and textile coatings disclosed herein and provided in the form of a concentrate are a liquid at ambient conditions, and are more preferably a water in oil emulsion. Without wishing to be bound by theory, the HSAEMs and essential oil(s) when diluted and applied to the textile materials synergistically and advantageously interact with one another to reduce (by binding and neutralizing) certain organic malodors (i.e., odors often associated with human body odor including odorous human secretions and/or by-product produced by bacteria and/or yeast found on the human body) on textile materials for a prolonged period of time. The organic malodors include, for example, isovaleric acid, ammonia, acetic acid, and nonenal.

The essential oils included within the odor control compositions and textile coatings (provided in the form of concentrate) are included at a concentration ranging from 25 wt % of the composition to 65 wt % of the composition in which any endpoint falling therein may serve as an endpoint for additional ranges, additional ranges may include, for example, from 25 wt % to 60 wt %, from 30 wt % to 55 wt %, from 35 wt % to 55 wt %, from 40 wt % to 50 wt %. In certain aspects, the essential oils include aloe oil, castor oil, hemp seed oil, flax seed oil, canola oil, or any combination thereof. In certain aspects, the essential oil comprises at least two of aloe oil, castor oil, hemp seed oil, canola oil, and flax seed oil. In certain aspects, essential oil comprises at least three of aloe oil, castor oil, hemp seed oil, canola oil, and flax seed oil. When there are two essential oils present in the compositions, the oils may be present in a 5:1 to 1:5 ratio, a 3:1 to 1:3 ratio, or a 1:1 ratio. In certain aspects, the essential oil comprises aloe oil, castor oil, hemp seed oil, and flax seed oil. When applied to a textile material, each of the above-mentioned essential oils function to control odor by binding and/or neutralizing the organic molecules while concurrently functioning to adhere the HSAEM to the textile material.

Moreover, the HSAEMs included within the odor control compositions and textile coatings (provided in the form of concentrate) are included at a concentration ranging from 10 wt % of the composition to 50 wt % of the composition in which any endpoint falling therein may serve as an endpoint for additional ranges, additional ranges may include, for example, from 15 wt % to 50 wt %, from 20 wt % to 45 wt %, from 25 wt % to 40 wt %, from 30 wt % to 40 wt %. In certain aspects, the HSAEM is a clay material in which the clay material is a zeolite present in the concentrated compositions at the above-mentioned concentrations. Clay materials and zeolites are preferred due to their high surface area and capabilities to bind and neutralize the disclosed malodor molecules. In certain preferred aspects, the zeolite is chabazite and/or pentasil. Moreover, the HSAEMs disclosed herein are provided in the disclosed compositions as polydisperse particles ranging from 0.1 μm to 10 μm, more preferably from 1 μm to 5 μm, which advantageously allows the disclosed essential oils to permanently fix and/or cure the HSAEMs on the textile material. HSAEM particles exceeding the above-mentioned highest endpoint should be avoided as they are coarse and will be easily worn off of the textile material post-application thereto (as well as negatively impact the haptic/tactile feel), which will disadvantageously result in reduced odor control; and HSAEM particles falling below the above-mentioned lowest endpoint due to regulatory restriction prohibiting the use of nanoparticles on textile materials (due to potential skin absorption and toxicological effects).

In certain aspects, the essential oil and HSAEM are present in at a ratio of 3:1 to 1:3, 2:1 to 1:2 more preferably 1.5:1 to 1:1.5, most preferably 1:1 relative to one another. The essential oils disclosed herein, when present in the above-mentioned concentrations and/or ratios, advantageously function to adhere (e.g., permanently adhere and/or adhere for a prolong period of time—25 wash cycles, months or years) the HSAEM(s) to the textile material (during and post-application of the compositions to the textile material) thereby achieving the desired odor control and reduction by binding and/or neutralizing the organic molecules while concurrently functioning to adhere the HSAEM to the textile material.

### Method of Applying The Odor Control And Textile Coating Compositions

As further discussed and shown in the Working Examples, the odor control compositions and textile coatings provided in the form of concentrates may be applied (permanently applied) to a desired textile material via a padding or exhausting method. When proceeding in this manner, approximately 2 wt % to 5 wt % of the above-mentioned odor control compositions and textile coatings provided in the form of concentrate is diluted in water (e.g., 95 wt % to 98 wt %) and this dilution is subsequently applied to the textile material via the padding method or the exhausting method.

For example, when using the padding method, the pad bath is made by combining the concentrate of the odor control/textile coating composition and water, which is approximately 2-5% concentrate of the composition and 95-98% water. The pad bath is padded onto the desired textile material and is subsequently cured/dried at 140° C. in an infrared (IR) drier for 1 minute to obtain a dry textile material that neutralizes odor and/or organic malodor molecules (secreted and/or excreted from humans) on the textile material for a prolonged period of time (e.g., 25 wash cycles, several months, or up to a year).

For example, in the exhausting method, the exhaustion bath is made by combining the concentrate of the odor control/textile composition and water, which is approximately 0.2-0.5% concentrate and 99.5-99.8% water. The exhaustion may be performed in a beaker dyeing machine that includes the desired textile material, e.g., Lobomat BFA-24 Werner Mathis AG. The exhaustion conditions may include the following: liquor ratio of 10 to 1, exhaustion temperature at 130° C., dwell time at the exhaustion temperature was 40 minutes, and the heating and cooling rate was 2° C./minute. After the exhaustion, the treated textile materials were rinsed and then cured/dried in an IR drier at 140° C. for 1 minute to obtain a dry textile material having that neutralizes odor and/or organic malodor molecules (secreted and/or excreted from humans) on the textile material for a prolonged period of time (e.g., 25 wash cycles, several months, or up to a year).

### Textile Materials Having The Odor Control And Textile Coating Compositions

Further disclosed herein are the dry textile material(s) having one of the above-mentioned compositions applied thereon via, for example, the above-mentioned padding or exhausting methods. The dry textile material having the odor control/textile coating composition applied thereon binds and neutralizes organic malodor molecules on the dry textile material for a prolonged period of time especially when compared with the same dry textile materials not treated with the compositions disclosed herein (and/or having the compositions disclosed herein applied thereon).

In this aspect, the dry textile material includes the essential oil applied thereon at a concentration ranging from 1.5% percent weight on fabric (owf) to 6.5% owf, more preferably 1.75% owf to 5% owf, and most preferably 2% owf to 3% owf, and the HSAEM is applied on the dry textile material at a concentration ranging from 1.5% percent weight on fabric (owf) to 6.5% owf, more preferably 1.75% owf to 5% owf, and most preferably 2% owf to 3% owf. In this aspect, the essential oil and HSAEM are present on the dry textile material at a ratio of 3:1 to 1:3, 2:1 to 1:2 more preferably 1.5:1 to 1:1.5, most preferably 1:1.

In view of the above mentioned concentrations and ratios, the dry textile material and/or the composition applied thereon controls odors for prolonged periods of time by binding and neutralizing ammonia, acetic acid, and isovaleric acid on the textile material especially when compared to an untreated textile material. In particular, the dry textile material binds and neutralizes 90% to 100% and more preferably 95% to 100% of ammonia when compared to an untreated textile material, and/or the dry textile material binds and neutralizes 20% to 70% and more preferably 30% to 70% of acetic acid when compared to an untreated textile material, and/or the dry textile material binds and neutralizes 20% to 70% and more preferably 30% to 70% of isovaleric acid when compared to an untreated textile material.

In certain preferred aspects, the textile materials disclosed herein, including the dry textile materials, are either woven or non-woven textile materials. In certain preferred embodiments, the textile materials and/or dry textile materials are knitted/woven fabrics, including, polyester, nylon, rayon, cotton, or any combination thereof. In certain aspects, the textile material/dry textile material includes a fabric weight ranging from 20-400 gsm (grams per square meter). It should be further appreciated that heavier weighted fabrics will absorb more of the above disclosed compositions (e.g., during and post-padding and/or during and post-exhaustion) resulting in a dry textile material having better odor control/capture for prolonged periods of time.

### Working Examples

Table 1 provides exemplary compositions (i.e., Compositions 1-7) of those disclosed herein as well as comparative formulations (i.e., Comparative Formulations 1-6). Each composition disclosed within Table 1 (Comparative compositions 1-6 and Compositions 1-7) were applied to polyester substrates/fabrics via padding or exhaustion methods with each polyester substrate/fabric having a weight of 106 gsm. As shown in Table 1, compositions 1-6, which include aloe vera oil and chabazite applied to a textile material using either padding or exhaustion, demonstrate at least one of a much higher ammonia reduction, isovaleric acid reduction, and/or acetic acid reduction when compared with the corresponding Comparative Formulations 1-4, which include aloe vera alone at various concentrations and applied to the same textile material using either padding or exhaustion. As specifically shown in Table 1, Compositions 1-6 each showed a marked improvement in ammonia reduction on the textile material when compared with textiles treated with aloe vera oil alone. Comparative Formulations 5 and 6, which only include pentasil (Comparative Formulation 5) or pentasil, baking soda, and citric acid (Comparative Formulation 6), demonstrate a lower isovaleric acid reduction ammonia reduction when compared to Composition 7 (pentasil, aloe oil, dissolvineR (Dissolvine GL-47-S Safety Data Sheet, Version 1, Revision Date Nov. 18, 2020, Print Date Apr. 27, 2021, p. 1-12), and citric acid). Isovaleric acid reduction was measured by ISO 17299-3. Ammonia reduction was measured by ISO 17299-2. Acetic acid reduction was measured by ISO 17299-2, and nonenal reduction was measured by ISO 17299-3. Comparative examples 1 and 2 and Compositions 1-3 each used Connoils Aloe Vera Oil (Safety Data Sheet last revised Oct. 6, 2021, pages 1-4, which includes Aloe Barbadensis Leaf Juice (CAS No. 94349-62-9) at 99.52 wt % and potassium sorbate (CAS No. 590-00-1) at 0.2 wt %, sodium benzoate (CAS No. 532-32-1) at 0.1 wt % and citric acid (CAS No. 77-92-9) at 0.18 wt %). Comparative examples 3 and 4 and composition 4 utilize Biovera® Aloe Vera Oil (Safety Data Sheet Issue date Dec. 11, 2015, pages 1-8, including canola oil (CAS No. 120962-03-0) and aloe barbadensis leaf extract (CAS No. 85507-69-3).

| | Level | | Level | Method of | | ISO IVA | MBI | Ammonia | Acetic Acid | Nonenal |
|---|---|---|---|---|---|---|---|---|---|---|
| Additive #1 | (owf) | Additive #2 | (owf) | Applying | Color | Reduction | IVA | Reduction | Reduction | Reduction |
| Comparative 1: | 2.0% | | | Padded | White | 57% | — | 32% | 54% | 76% |
| Connoils Aloe Vera Oil | | | | | | | | | | |
| Comparative 2: | 10.0% | | | Exhausted | White | | 71% | 7% | | |
| Connoils Aloe Vera Oil | | | | | | | | | | |
| Composition 1: | 2.5% | Chabazite | 2.5% | Exhausted | White | 24% | | 100% | 34% | Submitted |
| Connoils Aloe Vera Oil | | | | | | | | | | |
| Composition 2: | 2.5% | Chabazite/ | 2.5% | Exhausted | White | 28% | | 100% | 27% | Submitted |
| Connoils Aloe Vera Oil | | Arginine | | | | | | | | |
| Composition 3: | 3.0% | Chabazite | 2.0% | Exhausted | White | | 74% | Submitted | | |
| Aloe Vera Oil | | | | | | | | | | |
| Comparative 3: | 2.0% | | | Padded | White | 75% | — | 26% | Submitted | Submitted |
| Biovera Aloe Oil | | | | | | | | | | |
| Comparative 4: | 10.0% | | | Exhausted | White | 40% | — | 12% | Submitted | 97% |
| Biovera Aloe Oil | | | | | | | | | | |
| Composition 4: | 2.0% | Chabazite | 2.0% | Padded | White | 61% | — | 100% | Submitted | Submitted |
| Biovera Aloe Oil | | | | | | | | | | |
| Composition 5: Aloe | 2.0% | Hemp Oil | 0.5% | Padded | White | 71% | | 100% | 7% | Submitted |
| Vera Oil/Chabazite | | | | | | | | | | |
| Composition 6: Aloe | 2.0% | Castor Oil | 0.5% | Padded | White | 64% | | 100% | 40% | Submitted |
| Vera Oil/Chabazite | | | | | | | | | | |
| Comparative 5: | 5.0% | | | Exhausted | White | 41% | — | 26% | 41% | Submitted |
| Pentasil | | | | | | | | | | |
| Comparative 6: | 2.0% | Citric Acid | 1.0% | Exhausted | White | 28% | — | 13% | Prepared | 31% |
| Pentasil/Baking Soda | | | | | | | | | | |
| Composition 7: | 3.0% | Biovera Aloe | 1.0% | Exhausted | White | 55% | — | 79% | Submitted | Submitted |
| Pentasil | | Oil/Dissolvine/ | | | | | | | | |
| | | Citric Acid | | | | | | | | |

The padding method used to apply Comparative Examples 1 and 3 as well as Compositions 4-6 to the textile material in Table 1 includes the following: The pad bath is made from combining the concentrate of the solution and water, which is approximately 2-5% concentrate and 95-98% water. The pad bath was then padded onto the polyester and nylon fabrics with a laboratory padding machine. After the padding, fabrics were cured/dried at 140° C. using an IR drier for 1 minute.

The exhausting method used to apply Comparative Examples 2 and 4 as well as Compositions 1-3 to the textile material in Table 1 include the following: the exhaustion bath is made from combining the concentrate of the solution and water which is approximately 0.2-0.5% concentrate and 99.5-99.8% water. The exhaustion was performed in a beaker dyeing machine, Lobomat BFA-24 Werner Mathis AG. The exhaustion conditions were the following: liquor ratio was 10 to 1, exhaustion temperature was 130° C., dwell time at the exhaustion temperature was 40 minutes, and the heating and cooling rate was 2° C./minute. After the exhaustion fabrics were rinsed and then cured/dried in an IR drier at 140° C. for 1 minute.

The foregoing description provides embodiments of the invention by way of example only. It is envisioned that other embodiments may perform similar functions and/or achieve similar results. Any and all such equivalent embodiments and examples are within the scope of the present invention and are intended to be covered by the appended claims.

What is claimed is:

1. An odor control composition comprising:
(a) an essential oil that binds and/or neutralizes organic malodor molecules; and
(b) a high surface area essential mineral (HSAEM) that binds and/or neutralize organic malodor molecules.

2. The odor control composition of claim 1, wherein the essential oil is present in the odor control composition at a concentration ranging from 25 wt % to 65 wt %.

3. The odor control composition of claim 1, wherein the HSAEM is present in the odor control composition at a concentration ranging from 10 wt % to 50 wt %.

4. The odor control composition of claim 1, wherein the essential oil and HSAEM are present in at a ratio of 2:1 to 1:2 and/or water is present in the composition at a concentration from 10 wt % to 30 wt %.

5. The odor control composition of claim 1, wherein the essential oil comprises aloe oil, castor oil, hemp seed oil, flax seed oil, or any combination thereof.

6. The odor control composition of claim 1, wherein the essential oil comprises at least two of aloe oil, castor oil, hemp seed oil, and flax seed oil.

7. The odor control composition of claim 1, wherein the essential oil comprises at least three of aloe oil, castor oil, hemp seed oil, and flax seed oil.

8. The odor control composition of claim 1 wherein the essential oil comprises aloe oil, castor oil, hemp seed oil, and flax seed oil.

9. The odor control composition of claim 1, wherein the HSAEM is a zeolite.

10. The odor control composition of claim 1, wherein the HSAEM is chabazite, pentasil, or any combination thereof.

11. The odor control composition of claim 1, wherein the composition is a liquid.

12. The odor control composition of claim 1, wherein the composition is a water in oil emulsion.

13. The odor control composition of claim 1, wherein the organic malodor molecules comprise ammonia, acetic acid, isovaleric acid, or any combination thereof.

14. The odor control composition of claim 1, wherein the organic malodor molecules comprise at least two of ammonia, acetic acid, and isovaleric acid.

15. The odor control composition of claim 1, wherein the organic malodor molecules comprises ammonia, acetic acid, and isovaleric acid.

16. A textile coating composition comprising:
(a) an essential oil that binds and neutralizes organic odor molecules; and
(b) a high surface area essential mineral that binds and neutralizes organic odor molecules.

17. A dry textile material having the composition of claim 16 applied thereon, wherein the dry textile material and/or the composition applied thereon binds and neutralizes organic malodor molecules on the dry textile material for a prolonged period of time.

18. The dry textile material of claim 17, wherein the essential oil applied on the textile ranges from 1.5% percent weight on fabric (owf) to 6.5% owf, and the HSAEM applied on the textile ranges from 1.5% percent weight on fabric (owf) to 6.5% owf.

19. The dry textile material of claim 17, wherein the essential oil and HSAEM are present in at a ratio of 2:1 to 1:2 and/or water is present in the composition at a concentration from 10 wt % to 30 wt %.

20. The dry textile material of claim 17, wherein the dry textile material binds and neutralizes ammonia, acetic acid, and isovaleric acid on the textile material when compared to an untreated textile material.

21. The dry textile material of claim 20, wherein the dry textile material binds and neutralizes 90% to 100% of ammonia when compared to an untreated textile material, and/or the dry textile material binds and neutralizes 20% to 50% of acetic acid when compared to an untreated textile material, and/or the dry textile material binds and neutralizes 20% to 50% of isovaleric acid when compared to an untreated textile material

22. A method of applying an odor control composition of claim 1 to a textile material comprising:
applying the odor control composition to a textile material, wherein the odor control composition comprises (a) an essential oil at an effective amount that binds and neutralizes organic odor molecules; and (b) a high surface area essential mineral (HSAEM) at an effective amount that binds and neutralizes odor molecules.

23. The method according to claim 22, wherein applying the odor control composition to the textile material comprises exhausting.

24. The method according to claim 22, wherein applying the odor control composition to the textile material comprises padding.

25. The method according to claim 22, wherein the textile material has the odor control composition dried and/or cured thereon and binds and neutralizes organic malodor molecules on the dry textile material for a prolonged period of time.

26. The method according to claim 25, wherein the prolonged period of time comprises ten wash cycles, 25 wash cycles, for 3 months, for one year, or any combination thereof.
